(19) United States
(12) Patent Application Publication
(10) Pub. No.: US 2022/0196669 A1
(43) Pub. Date: Jun. 23, 2022

(54) METHOD FOR DETECTING OR MONITORING THE DEVELOPMENT OF A CHRONIC PROLIFERATIVE DISEASE BY IMMUNOASSAY

(71) Applicant: POLYNEUROS, SAINT JEAN D'ILLAC (FR)

(72) Inventors: Michel GEFFARD, Talence (FR); Jean-Pascal ZAMBAUX, Bouliac (FR)

(21) Appl. No.: 17/600,728

(22) PCT Filed: Apr. 1, 2020
(86) PCT No.: PCT/EP2020/059229

§ 371 Date: Oct. 1, 2021

(30) Foreign Application Priority Data
Apr. 1, 2019 (FR) FR1903450

(51) Int. Cl. G01N 33/574; G01N 33/53

(52) U.S. Cl. CPC G01N 33/57488; G01N 21/31; G01N 33/5304

(57) ABSTRACT

The invention relates to an ex vivo method for detecting or monitoring the progression of a chronic proliferative disease, in a sample of human or animal serum or plasma, by immunoassay for the presence of antibodies in the sample, including at least one or several antibodies directed against benzo(a)pyrene.

The invention also relates to a kit for implementing such a method.

### CROSS-REFERENCE TO RELATED APPLICATIONS

This application is a U.S. National Stage Application of PCT/EP2020/059229 assigned the international filing date of Apr. 1, 2020 and claiming the benefit of priority from French patent application FR1903450 filed Apr. 1, 2019, the disclosure of these applications is herein incorporated by reference.

### TECHNICAL FIELD

The invention relates to the ex vivo detection and monitoring of the progression of chronic proliferative diseases in humans or animals.

### BACKGROUND

The excessive proliferation of human or animal cells contributes to the pathogenesis of several chronic diseases called proliferative diseases, such as cancers, lymphomas, myelomas and leukemias. These pathologies involve a cell transformation that is generally associated with inflammatory processes and the passage of bacterial, viral and endotoxin components through the mucous membranes. In addition to having the characteristic of being proliferative, degenerative and/or autoimmune processes can develop during the progression of the disease. This is called comorbidity, which corresponds to the result of the association between proliferative disease and autoimmune and/or degenerative disease.

The therapeutic solutions provided for proliferative diseases are often immunosuppressive treatments such as chemotherapy or radiotherapy, which are not specific enough, or immunoregulatory treatments (monoclonal antibodies), which have a limited efficacy over time and are limited to only some patients. In addition to the ineffectiveness of some of these treatments, they are generally toxic and have significant side effects.

Furthermore, treatments are often ineffective in the very frequent cases where proliferative diseases are diagnosed late. Indeed, while several diagnostic tests exist, they are often expensive, complicated to implement and not always reliable.

In addition, since these pathologies are chronic, they require regular monitoring of the progression of the disease, and there is currently no simple and reliable solution that allows such monitoring.

### SUMMARY

The objective of the invention is to overcome the drawbacks of the prior art by proposing a method for both detecting and monitoring the progression of a chronic proliferative disease in humans or animals, which is simple to implement, minimally invasive, fast, efficient and reliable.

To this end, the object of the invention is an ex vivo method for detecting or monitoring the progression of a chronic proliferative disease, in a sample of human or animal biological fluid, preferably of human or animal plasma and/or serum, by immunoassay for the presence of antibodies in the sample, including at least one antibody directed against benzo(a)pyrene.

In addition to assaying at least one antibody directed against benzo(a)pyrene, the method according to the invention can also comprise immunoassay for the presence of at least one other antibody. This may in particular be at least one antibody directed against phosphatidylinositol and/or at least one antibody directed against azelaic acid.

Advantageously, this method is simple to carry out, since it is carried out on a sample of biological fluid and it implements conventional immunoassay techniques. The immunoassay method mimics ex vivo what happens in vivo where antigens are bound to immunoglobulins (antigens).

The method according to the invention can be implemented using a kit that constitutes another object of the invention.

Other features and advantages of the invention will emerge from the detailed description that follows.

### DESCRIPTION OF THE FIGURES

FIG. 1 shows the results of the detection of circulating antibodies directed against Benzo[a]pyrene (BAP) in IgA, dibenz[a,c]anthracene (DBA) in IgA, 7,12-dimethylbenza[a]anthracene (DMBA) in IgA, 1,2-benzanthracene (BA) in IgA, benzo(ghi)perylene(B(ghi)P in IgA and the negative control N-acetylcysteine (NAC) in IgA, during cancer progression, as a distribution of a population of cancer patients; the midpoint represents 50% of the population.

FIG. 2 shows the results of the detection and the progression of circulating antibodies directed against Benzo[a]pyrene (BAP) in IgA, dibenz[a,c]anthracene (DBA) in IgA, 7,12-dimethylbenza[a]anthracene (DMBA) in IgA, 1,2-benzanthracene (BA) in IgA, benzo(ghi)perylene(B(ghi)P in IgA and the negative control N-acetylcysteine (NAC) in IgA, during cancer progression.

### DEFINITIONS

Within the meaning of the invention, “circulating antibodies” refers to antibodies that are found in human or animal biological fluids, in particular in the blood, serum and/or plasma of humans or animals.

Within the meaning of the invention, “reagent blank” refers to a solution or a mixture containing all the reagents used during the method according to the invention, but which does not contain the sample to be analyzed. It allows a basic correction to be made to the results of the assay. It defines the background noise of the assay method.

Within the meaning of the invention, “biological fluid” refers to fluid from the body of a human being or of an animal, in particular blood, serum and/or plasma, cerebrospinal fluid, pleural fluids and intraperitoneal, intra-articular fluids, saliva or urine.

Within the meaning of the invention, “proliferative disease” refers to a disease characterized by abnormal and uncontrolled cell proliferation.

### DETAILED DESCRIPTION OF THE INVENTION

The object of the invention is therefore an ex vivo method for detecting or monitoring the progression of a chronic proliferative disease.

The disease can be any chronic proliferative disease, and it can in particular be cancer, especially lymphoma, myeloma or leukemia.

The method is carried out ex vivo, outside the human or animal body, in a sample of human or animal biological fluid, preferably of human or animal serum or plasma, which was taken before the implementation of the method. The sample of biological fluid is a sample that has been taken and stored according to the usual standards known to those skilled in the art.

The method according to the invention is carried out by immunoassay for the presence in the sample of circulating antibodies directed against specific antigens of chronic proliferative diseases.

The method can be any type of immunoassay method. It may in particular be an ELISA assay, strip tests, tests carried out using magnetic, latex or silica beads, or else a Western blot test. These tests can be implemented according to the knowledge of a person skilled in the art.

In the implementation of the method according to the invention:
The antigens used to implement the method are preferably antigens synthesized ex vivo;
The antigens used are preferentially coupled (conjugated) to a protein, such as bovine serum albumin for example, to facilitate the fix assay method); thus, an antibody directed against an antigen within the meaning of the present invention can mean (excluding bacteria) an antibody directed against a conjugated antigen, whether or not the expression “conjugated” is indicated after the antigen.
The secondary antibodies, called anti-isotypes, are preferentially conjugated to an enzyme such as peroxidase or alkaline phosphatase are diluted according to their isotype, in a diluting buffer, called storage buffer, containing stabilizing proteins. These antibodies are preferably known antibodies that can be purchased.

The method according to the invention preferably comprises an enzyme immunoassay based on the Elisa method. The immunoassay method can comprise the following steps:
the antigens against which the antibodies to be detected in the sample are directed are adsorbed (and optionally dried) on microtiter plates; this step consists in sensitizing microtiter plates with the antigen(s) against which the antibodies, the presence of which is to be detected in the sample, are directed.

The plates are then either used directly to implement the method, or dried and stored in a dry atmosphere, preferably at 4° C. and protected from light.
the sample to be tested is then preferably diluted; this dilution can be between 125 and 1000 times, in particular 150 to 1000 times;
similarly, preferably, test sera or plasmas, called internal standards, which are biological fluids from healthy subjects, preferably sera or plasma from healthy subjects (representative population matched with the patient population at least for sex and age), are used and preferably diluted; this dilution can be between 125 and 1000 times, in particular 150 to 1000 times;
the sample, a reagent blank and preferably also the internal standards, are distributed, preferably in duplicate, in wells of at least one sensitized microtiter plate,
preferably, the plate(s) are incubated; according to a particularly suitable embodiment, they are incubated between 1 h30 and 2 h00, preferably between 1 h45 and 2 h00, at a temperature of 35 to 39° C.,
preferably, the plate(s) are washed, that is to say, rinsed, preferably several times, with the aim of eliminating all the non-specific proteins and immunoglobulins;
then, anti-human or animal immunoglobulin antibodies, called secondary antibodies, are added that are directed against isotypes A, M or G for humans, A, M, G or E in animals; these secondary antibodies are antibodies directed against the immunoglobulins bound to the antigens present in the wells of the microtiter plates; the secondary antibodies are preferably coupled to an enzyme, and preferably to alkaline peroxidase or phosphatase, to allow a colorimetric reaction to be carried out,
preferably, the plates are incubated again for 1 hour to 2 hours at a temperature between 35 and 39° C.,
preferably, the plates are then washed again, that is to say, several rinses are carried out so as to eliminate what has not been specifically recognized,
preferably, a substrate of the enzyme (H₂O₂ for peroxidase or for alkaline phosphatase) is then applied with a chromogen; the objective of this step is to visualize the immunological reactions. The chromogen can for example be tetramethylbenzedine (TMB) or Diaminobenzedine (DAB); a colorimetric reaction appears; this is all the more intense when there are human or animal immunoglobulins fixed on the antigens present in the wells;
optionally, a new incubate is done for between 10 and 30 minutes at a temperature between 18 and 20° C., preferably protected from light; the reaction is then stopped very preferably in an acid solution;
the optical density of each well of the microtiter plates is then read, preferably using a spectrophotometer; the reading is preferably carried out at 450 nm with a correction alpha at 620 nm or at 650 nm; these optical densities are then preferably recorded in software.

According to a specific algorithm, the OD values of the patient's serum (human or animal) of all the specific markers defining his immunological profile are held up against the distribution of a population of patients suffering from a chronic proliferative pathology versus the distribution of a population of healthy controls. These population distributions depend on a Mann and Whitney U test with a probability associated with a Mann and Whitney U test (P value) threshold of 0.01, on a distribution by percentiles. It is thus possible to detect whether the human being or the animal to which the tested serum and/or plasma belongs is affected by a proliferative disease.

In addition, the method according to the invention also makes it possible to monitor the progression of the disease. The variation of the OD value for the sought antibody or antibodies makes it possible to verify the variation and the progression of the disease, defined by the P value of the Mann and Whitney U test:
If the P value is less than 0.01, defined by the Mann and Whitney U test, of the distribution of the patient population (human or animal) compared to the population distribution of healthy controls, then the level of circulating serum antibodies directed against a bacterial antigen and/or a neoantigen is significantly different from the level of circulating serum antibodies in a population of healthy controls.
If the P value is greater than 0.01, defined by the Mann and Whitney U test, of the distribution of the patient population (human or animal) compared to the population distribution of healthy controls, then the level of circulating serum antibodies directed against a bacterial antigen and/or a neoantigen is identical to the level of circulating serum antibodies in a population of healthy controls. The OD(s) obtained from the patient or animal are distributed by percentile according to the distribution chosen by the designed algorithm.

In the case of cancers, if the ODs are distributed over the distribution by percentiles of the patient population, then for the antibodies sought according to the invention, this means that it is a cancer and/or any other chronic proliferative pathology.

Assaying one or more specific antibodies also makes it possible to propose a treatment adapted as a function of the tested antibody or antibodies present in the sample.

According to a particular embodiment, the method according to the invention comprises at least the implementation of the following steps:
manufacturing at least one microtiter plate sensitized with the antigen(s) against which the antibodies, the presence of which is to be detected in the sample, are directed, or using at least one microtiter plate already sensitized with these antigens,
distributing the same volume (i.e. the same quantity) of internal standards, sample and reagent blank on the plate,
adding the secondary antibody or antibodies coupled to an enzyme,
adding an enzyme substrate and a chromogen, waiting for the coloring of the wells,
stopping the coloring reaction,
reading the optical density of the wells using a spectrophotometer at an appropriate wavelength.

In particular, a particularly suitable embodiment of the invention is a method that comprises at least implementing the following steps:
manufacturing at least one microtiter plate sensitized with the antigen(s) against which the antibodies, the presence of which is to be detected in the sample, are directed, or using at least one microtiter plate already sensitized with these antibodies,
diluting the sample to be tested and internal standards,
distributing, in duplicate on the plate, the same volume of diluted internal standards, diluted sample and reagent blank,
incubating,
washing,
adding the secondary antibody or antibodies coupled to peroxidase or alkaline phosphatase,
incubating,
washing,
adding a substrate and a chromogen,
stopping the reaction in acid solution
reading at 450 nm with a correction alpha at 620 nm or 650 nm.

The antibodies sought in the context of the method according to the invention, independent of the antigens against which they are directed, can be IgM (immunoglobulins of isotype M), IgA (immunoglobulins of isotype A), or IgG (immunoglobulins of isotype G), and in animals only, IgE (immunoglobulins of isotype E):
IgA antibodies: are the result of mucosal immune activation (intestinal, ENT, skin, bladder)
IgM antibodies: are the result of activation of the current immune system,
IgG antibodies: are the result of activation of the old immune system (memory immunity)
IgE antibodies (in animals only) are the result of the stimulation of mast cells, which release histamine, that is to say, they are the result of contact with allergenic antigens.

The method according to the invention is therefore carried out by immunoassay for the presence of antibodies in the sample, including at least one antibody directed against benzo(a)pyrene.

Indeed, according to the invention, the presence of antibodies directed against benzo(a)pyrene in a human or animal biological fluid indicates the presence of a chronic proliferative disease. According to the invention, these antibodies are involved in the physiological mechanisms at the origin of all chronic proliferative diseases.

Benzo(a)pyrene is a chemical carcinogen that binds to an intracyto plasma receptor called AhRmi, and benzo(a)pyrene is representative of all the ligands that bind to this receptor to activate it and cause the derepression of genes that cause overgrowth. The presence of benzo(a)pyrene indicates activation of the immune system by cellular components.

According to the invention, the presence in the biological fluid, in particular in the serum and/or in the plasma, of antibodies directed against benzo(a)pyrene, preferably IgA and/or IgG, necessarily means that the affected person or animal has a chronic proliferative disease.

The method according to the invention can also comprise the immunoassay for the presence of other antibodies in the sample in addition to the presence of antibodies directed against benzo(a)pyrene. In particular, the method according to the invention can also comprise the immunoassay for the presence in the sample of other antibodies, including at least one antibody chosen from:
antibodies directed against phosphatidylinositol,
antibodies directed against azelaic acid.

Azelaic acid is a product of the breakdown of unsaturated fatty acids. The presence of azelaic acid in humans or animals causes activation of the immune system by this product of lipoperoxidation. Indeed, the presence of azelaic acid in humans or animals is the result of oxidation and the intervention of free radicals OH, 0₂, NO and N0₂.

This means in particular that there is membrane destruction or an anarchic binding to other components of the self, and it therefore becomes immunogenic.

According to the invention, the presence in the biological fluid of antibodies directed against azelaic acid, in particular IgA and/or IgM and/or IgG, may mean that the concerned person or animal is suffering from a chronic proliferative disease, in particular when this presence is combined with that of benzo(a)pyrene.

The presence of phosphatidylinositol indicates activation of the immune system by cellular components.

Phosphatidylinositol is an intracellular mediator that causes a cascade of metabolic events during cell transformation. This metabolite is increased excessively (up to 200 times).

According to the invention, the presence in the biological fluid of antibodies directed against phosphatidylinositol, preferably IgA and/or IgM and/or IgG, can mean that the person or animal concerned is affected by a proliferative chronic disease, especially when this presence is combined with that of benzo(a)pyrene.

Preferably, the method according to the invention comprises the immunoassay for the presence of antibodies in the sample, including at least one of the following antibodies:
IgM and/or IgG and/or IgA (and/or IgE for animals) directed against benzo(a)pyrene.

It suffices for one of these antibodies to be present in the tested biological fluid sample, in particular in the tested serum and/or plasma, that is to say, for the test to be positive for at least one of these antibodies, in order for the person to have a chronic proliferative disease. According to one embodiment, the method according to the invention comprises the immunoassay for the presence of other antibodies in the sample, including at least one antibody chosen from the following antibodies:
IgM and/or IgG and/or IgA (and/or IgE for animals) directed against phosphatidylinositol,
IgM and/or IgG and/or IgA (and/or IgE for animals) directed against azelaic acid.

According to a variant, the method according to the invention comprises the immunoassay for the presence of antibodies in the sample, including at least the following antibodies:
IgM, IgG and IgA (and/or IgE for animals) directed against benzo(a)pyrene, and
IgM, IgG and IgA (and/or IgE for animals) directed against phosphatidylinositol, and
IgM, IgG and IgA (and/or IgE for animals) directed against azelaic acid.

The method according to the invention, in addition (either at the same time or in a separate test) to the immunoassay for the presence in the sample of at least one antibody directed against benzo(a)pyrene, and optionally of at least one antibody directed against phosphatidylinositol and optionally at least one antibody directed against azelaic acid, can also comprise the immunoassay for the presence in the sample of one or more other antibodies.

This may preferably be:
antibodies directed against neoantigens that are cofactors at the origin of chronic proliferative diseases, and which make it possible to confirm or refine the diagnosis and/or which make it possible to monitor the progression of the disease and/or which make it possible to offer a targeted treatment directed against the concerned antigen(s), and/or
antibodies directed against neoantigens that are consequences of chronic proliferative diseases, and which make it possible to monitor the progression of the disease and/or which make it possible to offer a targeted treatment directed against the concerned antigen(s).

According to a particular embodiment, the method according to the invention also comprises the immunoassay for the presence in the sample of at least one antibody directed against a neoantigen that is a cofactor at the origin of a chronic proliferative disease, and in particular of at least one of the following antibodies:
antibody or antibodies directed against palmitic acid,
antibody or antibodies directed against oleic acid,
antibody or antibodies directed against myristic acid,
antibody or antibodies directed against malondialdehyde.

The method according to the invention preferably comprises the immunoassay for the presence in the sample of at least two or of at least three or of at least four of the following antibodies or of all the following antibodies:
antibody or antibodies directed against palmitic acid,
antibody or antibodies directed against oleic acid,
antibody or antibodies directed against myristic acid,
antibody or antibodies directed against malondialdehyde.

Palmitic acid, oleic acid and myristic acid are acylating agents of membrane and cytoplasmic proteins. These compounds are not naturally immunogenic. Apart from oleic acid, these are saturated fatty acids that can yield unsaturated compounds that can be decomposed during lipoperoxidation mechanisms. They become so in the event of membrane destruction linked to the intervention of free radicals. The presence of one or more circulating antibodies directed against one of these acids therefore means that there has been a modification of the membrane structure of the proteins and/or of the lipoperoxidation mechanisms.

Malondialdehyde (or malonic aldehyde) is the consequence of oxidative stress. It results in particular from the action of reactive oxygen and NO derivatives on fatty acids. It reacts with deoxyadenosine and deoxyguanosine to form DNA adducts. Like azelaic acid, it is the result of oxidation and the intervention of free radicals on fatty acids. It is therefore also a marker for membrane destruction.

Preferably, the method according to the invention comprises the immunoassay for the presence of antibodies in the sample, including at least one of the following antibodies:
IgA, IgM and/or IgG (and/or IgE for animals) directed against palmitic acid,
IgA, IgM (and/or IgE for animals) directed against oleic acid,
IgA, IgM (and/or IgE for animals) directed against myristic acid,
IgA, IgM and/or IgG (and/or IgE for animals) directed against malondialdehyde,

According to a particularly suitable variant, the method according to the invention comprises the immunoassay for the presence of antibodies in the sample, including at least the following antibodies:
IgA, IgM and IgG (and/or IgE for animals) directed against palmitic acid, and
IgA, IgM (and/or IgE for animals) directed against oleic acid, and
IgA, IgM (and/or IgE for animals) directed against myristic acid, and
IgA, IgM and IgG (and/or IgE for animals) directed against malondialdehyde,

According to another particular embodiment, the method according to the invention also comprises the immunoassay for the presence in the sample of at least one antibody directed against an antigen that is a consequence of a chronic proliferative disease, and in particular of at least one antibody chosen from the following antibodies:
antibody or antibodies directed against 1,2-benzanthracene
antibody or antibodies directed against dibenz[a,c]anthracene,
antibody or antibodies directed against 7,12-dimethylbenza[a]anthracene
antibody or antibodies directed against benzo(ghi)perylene,
antibody or antibodies directed against cadaverine,
antibody or antibodies directed against putrescine—antibody or antibodies directed against Mycobacterium tuberculosis.

The method according to the invention preferably comprises the immunoassay for the presence in the sample of at least two or of all the following antibodies:
antibody or antibodies directed against 1,2-benzanthracene
antibody or antibodies directed against dibenz[a,c]anthracene,—antibody or antibodies directed against 7,12-dimethylbenza[a]anthracene
antibody or antibodies directed against benzo(ghi)perylene,
antibody or antibodies directed against cadaverine,
antibody or antibodies directed against putrescine,
antibody or antibodies directed against Mycobacterium tuberculosis.

1,2-Benzanthracene, dibenz[a,c]anthracene, 7,12-dimethylbenza[a]anthracene and benzo(ghi)perylene are carcinogenic polycyclic aromatic compounds that recognize the AhR receptor and environmental pollutants. These are molecules capable of inducing tumors. Cadaverine and putrescine are markers, that is to say, a consequence, of the hyperproduction of polyamines. They have a negative influence on patients.

Mycobacterium tuberculosis is a Gram-positive pathogenic bacterium responsible for tuberculosis. It is also a factor in patients suffering from other diseases, such as people or animals suffering from certain proliferative pathologies. It does not produce toxins and owes its pathogenicity to its ability to multiply. Lysis of the bacteria releases antigenic components that elicit an immune reaction inducing a state of hypersensitivity.

Preferably, the method according to the invention comprises the immunoassay for the presence of antibodies in the sample, including at least one of the following antibodies:
IgA, IgM and/or IgG (and/or IgE for animals) directed against 1,2-benzanthracene,
IgA, IgM and/or IgG (and/or IgE for animals) directed against dibenz[a,c]anthracene,
IgA, IgM and/or IgG (and/or IgE for animals) directed against 7,12-dimethylbenza[a]anthracene,
IgA, IgM and/or IgG (and/or IgE for animals) directed against benzo(ghi)perylene,
IgA, IgM and/or IgG (and/or IgE for animals) directed against cadaverine,
IgA, IgM and/or IgG (and/or IgE for animals) directed against putrescine,
IgA, IgM and/or IgG (and/or IgE for animals) directed against Mycobacterium tuberculosis.

According to a particularly suitable variant, the method according to the invention comprises the immunoassay for the presence of antibodies in the sample, including at least the following antibodies:
IgA, IgM and/or IgG (and/or IgE for animals) directed against 1,2-benzanthracene,
IgA, IgM and/or IgG (and/or IgE for animals) directed against dibenz[a,c]anthracene,
IgA, IgM and/or IgG (and/or IgE for animals) directed against 7,12-dimethylbenza[a]anthracene,
IgA, IgM and/or IgG (and/or IgE for animals) directed against benzo(ghi)perylene,
IgA, IgM and/or IgG (and/or IgE for animals) directed against cadaverine,
IgA, IgM and/or IgG (and/or IgE for animals) directed against putrescine,
IgA, IgM and/or IgG (and/or IgE for animals) directed against Mycobacterium tuberculosis.

To implement the method, the invention also relates to diagnostic kits.

In particular, the object of the invention is a kit for use thereof in the detection or monitoring of the progression of a chronic proliferative disease, in a sample of biological fluid, in particular in a sample of human or animal biological fluid comprising at least the antigen(s) against which the antibodies, the presence of which is to be detected in the sample, are directed, namely:
at least benzo(a)pyrene,
optionally at least one of the following antigens: palmitic acid, malondialdehyde, oleic acid, myristic acid, 1,2-benzanthracene, dibenz[a,c]anthracene, 7,12-dimethylbenza[a]anthracene, benzo(ghi)perylene, cadaverine, putrescine, Mycobacterium tuberculosis, and/or phosphatidylinositol and/or azelaic acid.

Preferably, the kit according to the invention comprises at least:
the antigen(s) against which the antibodies, the presence of which is to be detected in the sample, are directed, preferably coupled to a protein when they are not bacteria, and a microtiter plate intended to be sensitized with the antigen(s) against which the antibodies, the presence of which is to be detected in the sample, are directed, or a microtiter plate already sensitized with the antigen(s) against which the antibodies, the presence of which is to be detected in the sample, are directed, or a strip or stick already sensitized with the antigen(s) against which the antibodies, the presence of which is to be detected in the sample, are directed, and
the standards making it possible to assess the quality of the test and the distribution of the control population, and/or
buffers and solutions suitable for performing an ELISA test, and/or
the secondary antibody or antibodies in solution, said secondary antibody or antibodies corresponding to the isotypes of the primary antibodies, the presence of which is to be detected in the sample, and/or
dilution and washing buffers, and/or
development buffers, and/or
a stop solution.

The invention is now illustrated by examples and results of implementing methods according to the invention.

For each example and associated results, the protocol was as follows:
collection of the patient's plasma
implementation of the method according to the invention
summary of the results obtained in a table to visualize the detection of circulating antibodies and monitor their progression

Each example makes it possible to make the link between the distribution of the population affected by a chronic proliferative pathology compared to the distribution of a control population.

### EXAMPLES

### Example 1: Results of the Implementation of a Method According to the invention on a Person Suffering from Cancer

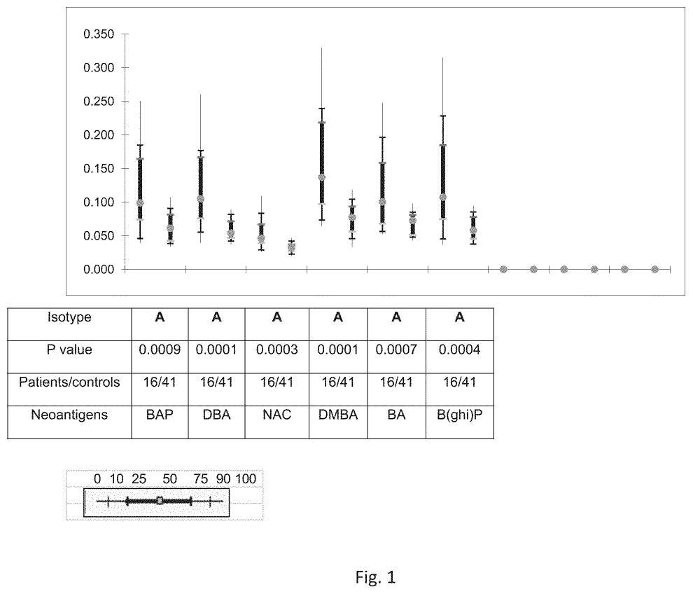

The results of the detection of circulating antibodies directed against Benzo[a]pyrene (BAP) in IgA, dibenz[a,c]anthracene (DBA) in IgA, 7,12-dimethylbenza[a]anthracene (DMBA) in IgA, 1,2-benzanthracene (BA) in IgA, benzo(ghi)perylene(B(ghi)P in IgA and the negative control N-acetylcysteine (NAC) in IgA, during the progression of the disease are shown in FIG. 1, as a distribution of a population of cancer patients; the midpoint represents 50% of the population.

### Example 2: Results of the Implementation of a Method According to the Invention on a Person Suffering from Cancer

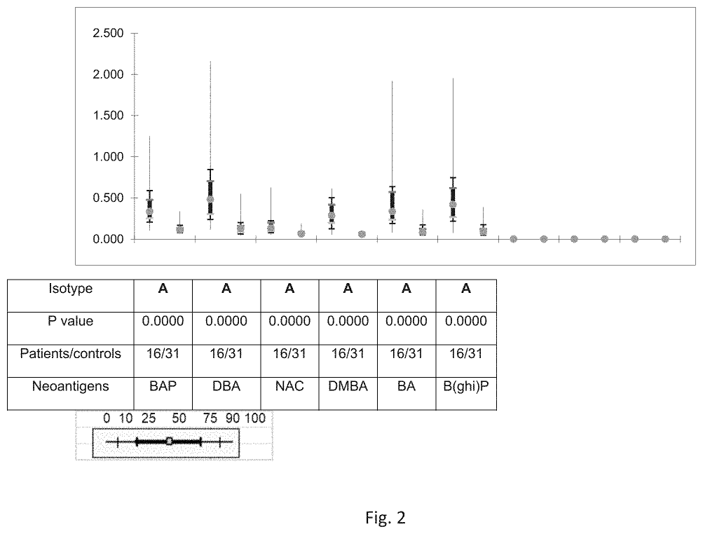

The results of the detection and the progression of circulating antibodies directed against Benzo[a]pyrene (BAP) in IgA, dibenz[a,c]anthracene (DBA) in IgA, 7,12-dimethylbenza[a]anthracene (DMBA) in IgA, 1,2-benzanthracene (BA) in IgA, benzo(ghi)perylene(B(ghi)P in IgA and the negative control N-acetylcysteine (NAC) in IgA, during the progression of the disease are shown in FIG. 2.

These various examples show in particular that populations suffering from chronic proliferative diseases always exhibit circulating antibodies directed against at least one or more antibodies directed against Benzo(a)pyrene.

The method according to the invention also makes it possible to monitor the progression of the disease.

1. An ex vivo method for detecting or monitoring progression of a chronic proliferative disease, in a sample of human or animal biological fluid, by immunoassay for the presence of antibodies in the sample, including at least one antibody directed against benzo(a)pyrene.

2. The ex vivo method of detecting or monitoring progression of a chronic proliferative disease according to claim 1, characterized in that the sample is a sample of human or animal serum or plasma.

3. The ex vivo method for detecting or monitoring the progression of a chronic proliferative disease according to claim 1, characterized in that it also comprises the immunoassay for the presence in the sample of at least one of the following antibodies:
at least one antibody directed against phosphatidylinositol, and/or
at least one antibody directed against azelaic acid.

4. The ex vivo method for detecting or monitoring the progression of a chronic proliferative disease according to claim 1, characterized in that it also comprises the immunoassay for the presence in the sample of at least one of the following antibodies:
antibody or antibodies directed against palmitic acid,
antibody or antibodies directed against malondialdehyde
antibody or antibodies directed against oleic acid,
antibody or antibodies directed against myristic acid.

5. The ex vivo method for detecting or monitoring the progression of a chronic proliferative disease according to claim 1, characterized in that it also comprises the immunoassay for the presence in the sample of at least one of the following antibodies:
antibody or antibodies directed against 1,2-benzanthracene,
antibody or antibodies directed against dibenz[a,c] anthracene,
antibody or antibodies directed against 7,12-dimethylbenza[a] anthracene,
antibody or antibodies directed against benzo(ghi)perylene,
antibody or antibodies directed against cadaverine,
antibody or antibodies directed against putrescine,
antibody or antibodies directed against Mycobacterium tuberculosis.

6. The ex vivo method for detecting or monitoring the progression of a chronic proliferative disease according to claim 1, characterized in that the antibodies are immunoglobulins A and/or immunoglobulins G and/or immunoglobulins M and/or, for animals only, immunoglobulins E.

7. The ex vivo method for detecting or monitoring the progression of a chronic proliferative disease according to claim 1, characterized in that said disease is chosen from cancers.

8. The ex vivo method for detecting or monitoring the progression of a chronic proliferative disease according to claim 1, characterized in that the disease is chosen from lymphomas, myelomas and leukemias.

9. The ex vivo method for detecting or monitoring the progression of a chronic disease according to claim 1, characterized in that the immunoassay is carried out by implementing an immuno-enzymatic method.

10. The ex vivo method for detecting or monitoring the progression of a chronic disease according to claim 1, characterized in that it comprises at least the implementation of the following steps:
manufacturing at least one microtiter plate sensitized with the neoantigen(s) against which the antibodies, the presence of which is to be detected in the sample, are directed, or using at least one microtiter plate already sensitized with these antigens,
distributing the same volume of internal standards, sample and reagent blank on the plate,
adding the secondary antibody or antibodies coupled to an enzyme,
adding an enzyme substrate and a chromogen, waiting for the coloring of the wells,
stopping the coloring reaction, and
reading the optical density of the wells using a spectrophotometer at an appropriate wavelength.

11. Ex vivo method for detecting or monitoring the progression of a chronic disease according to the preceding claim, characterized in that it comprises at least the implementation of the following steps:
manufacturing at least one microtiter plate sensitized with the neoantigen(s) against which the antibodies, the presence of which is to be detected in the sample, are directed, or using at least one microtiter plate already sensitized with these antigens,
diluting the sample to be tested and internal standards,
distributing, in duplicate on the plate, the same volume of diluted internal standards, diluted sample and reagent blank,
incubating,
washing,
adding the secondary antibody or antibodies coupled to peroxidase or alkaline phosphatase,
incubating,
washing,
adding a substrate and a chromogen,
stopping the reaction in acid solution, and
reading at 450 nm with a correction alpha at 620 nm.

12. A kit for use in a method for detecting or monitoring the progression of a chronic proliferative disease according to claim 1, characterized in that it comprises at least the neoantigen(s) against which the antibodies, the presence of which is to be detected in the sample, are directed.

13. The kit according to claim 12, characterized in that it comprises at least:
a microtiter plate sensitized with the neoantigen(s) against which the antibodies, the presence of which is to be detected in the sample, are directed,
buffers and solutions, and
the secondary antibody or antibodies in solution, secondary antibodies corresponding to the antibodies whereof the presence is to be detected in the sample.
